SEQUENCE LISTING (19 sequences)

SEQ ID NO: 1 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 54 aa, Artificial Sequence
  Synthetic SCR7
CYFPYLENGYNQNYGRKFVQGKSIDVACHPGYALPKAQTTVTCMENGWSPTPRC

SEQ ID NO: 3 - DNA, 162 bp, Artificial Sequence
  Synthetic SCR7 -DNA
tgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgc

SEQ ID NO: 4 - protein, 458 aa, Artificial Sequence
  Synthetic SCR1-7
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGSHHHHHH

SEQ ID NO: 5 - DNA, 1440 bp, Artificial Sequence
  Synthetic SCR1-7 DNA
atgcgatggtcttggatttttctgctgctgctgtctatcacttctgctaacgctgaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaactggcgctagcggcagccaccaccatcaccaccattgataaggatccagacatgataagatacattgatgagtttggacaaaccacaactagaatgcagtg

SEQ ID NO: 6 - protein, 1102 aa, Artificial Sequence
  Synthetic SCR 1-18
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQC

SEQ ID NO: 7 - DNA, 3306 bp, Artificial Sequence
  Synthetic SCR1-18 DNA
atgcgatggtcttggatttttctgctgctgctgtctatcacttctgctaacgctgaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgc

SEQ ID NO: 8 - protein, 874 aa, Artificial Sequence
  Synthetic SCR 7-20
MRWSWIFLLLLSITSANAASGCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 9 - DNA, 2622 bp, Artificial Sequence
  Synthetic SCR 7-20 DNA
atgcgatggtcttggatttttctgctgctgctgtctatcacttctgctaacgctgctagcggctgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgagaactacttgttgggacgggaaactggaataccctacttgtgctaagagaggcggaagcggccaccaccatcaccaccat

SEQ ID NO: 10 - protein, 744 aa, Artificial Sequence
  Synthetic SCR 7-18
MRWSWIFLLLLSITSANAEDCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCGGSGHHHHHH

SEQ ID NO: 11 - DNA, 2232 bp, Artificial Sequence
  Synthetic SCR 7-18 DNA
atgcgatggtcttggatttttctgctgctgctgtctatcacttctgctaacgctgaggactgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcggcggaagcggccaccaccatcaccaccat

SEQ ID NO: 12 - protein, 211 aa, Artificial Sequence
  Synthetic SCR 6-8
MRWSWIFLLLLSITSANAEDCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCGGSGHHHHHH

SEQ ID NO: 13 - DNA, 633 bp, Artificial Sequence
  Synthetic SCR 6-8 DNA
atgcgatggtcttggatttttctgctgctgctgtctatcacttctgctaacgctgaggactgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcggcggaagcggccaccaccatcaccaccat

SEQ ID NO: 14 - protein, 277 aa, Artificial Sequence
  Synthetic SCR 1-4
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGSHHHHHH

SEQ ID NO: 15 - DNA, 831 bp, Artificial Sequence
  Synthetic SCR 1-4 DNA
atgcgatggtcttggatttttctgctgctgctgtctatcacttctgctaacgctgaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatctggcgctagcggcagccaccaccatcaccaccat

SEQ ID NO: 16 - protein, 399 aa, Artificial Sequence
  Synthetic SCR 1-6
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKGASGSHHHHHH

SEQ ID NO: 17 - DNA, 1197 bp, Artificial Sequence
  Synthetic SCR 1-6 DNA
atgcgatggtcttggatttttctgctgctgctgtctatcacttctgctaacgctgaggactgtaacgaactgcccccacgaaggaataccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaaggcgctagcggcagccaccaccatcaccaccat

SEQ ID NO: 18 - protein, 815 aa, Artificial Sequence
  Synthetic SCR 8-20
MRWSWIFLLLLSITSANAASGCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 19 - DNA, 2445 bp, Artificial Sequence
  Synthetic SCR 8-20 DNA
atgcgatggtcttggatttttctgctgctgctgtctatcacttctgctaacgctgctagcggctgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgagaactacttgttgggacgggaaactggaataccctacttgtgctaagagaggcggaagcggccaccaccatcaccaccat

(19) United States
(12) Patent Application Publication
(10) Pub. No.: US 2019/0388506 A1
(43) Pub. Date: Dec. 26, 2019

(54) FACTOR H FRAGMENT FOR USE AS AN ANTI-ANGIOGENIC AGENT

(71) Applicants: UNIVERSITE PIERRE ET MARIE CURIE (PARIS 6), Paris (FR); INSERM (INSTITUT NATIONAL DE LA SANTÉ ET DE LA RECHERCHE MÉDICALE), Paris (FR); UNIVERSITE PARIS DIDEROT - PARIS 7, Paris (FR); UNIVERSITE PARIS DESCARTES, Paris (FR); LABORATOIRE FRANCAIS DU FRACTIONNEMENT ET DES BIOTECHNOLOGIES, Les Ulis (FR)

(72) Inventors: Virginie DINET, Breuillet (FR); Frédéric MASCARELLI, L'Hay les Roses (FR); Toufik ABACHE, Santes (FR); Sylvie JORIEUX, Lomme (FR)

(21) Appl. No.: 16/066,963

(22) PCT Filed: Dec. 30, 2016
(86) PCT No.: PCT/FR2016/053689

§ 371 Date: Jun. 28, 2018

(30) Foreign Application Priority Data
Dec. 30, 2015 (FR) 15 63465

(51) Int. Cl. A61K 38/17; A61P 9/10

(52) U.S. Cl. CPC A61K 38/1725; A61K 48/0058; A61P 9/10

(57) ABSTRACT

The invention relates to a complement factor H fragment for use in the treatment and/or prophylaxis of a disease involving neovascularization.

The present invention relates to a complement factor H (FH) fragment for use in the treatment and/or prophylaxis of a disease involving neovascularization.

Angiogenesis is a complex physiological mechanism that plays a major role in the genesis of the neovascularization involved in various diseases including solid tumor growth, atherosclerosis, rheumatoid arthritis, psoriasis, and intraocular neovascular diseases such as proliferative retinopathy or wet age-related macular degeneration (Wet AMD).

Inhibitors of vascular endothelial growth factor (VEGF) are used in the treatment of various eye diseases and cancers. Included among the currently available VEGF inhibitors are pegaptanib, bevacizumab, ranibizumab, or alibercept. Although these compounds constitute a breakthrough in the treatment of wet AMD, it has been found that the recovered sight obtained through these treatments is short-lived: the retinal edema secondary to the neovascularization recurs and retinal degeneration continues. In addition, 20-30% of patients have little response to these treatments (Rasmussen and Sander, 2014) and have residual exudative signs.

On the other hand, although excess VEGF has the effect of promoting neovascularization and the associated edema, complete inhibition can have a detrimental effect on the neural retina, the choroid, and the cells of the retinal pigment epithelium (RPE), and in particular its tight junctions which are critical to maintaining the integrity of the RPE. Indeed. VEGF is considered a trophic factor for the survival of RPE cells (Byeon et al., 2010), choriocapillaris maintenance (Saint-Geniez et al., 2009), and visual function (St. Geniez et al., 2008). Treatment with anti-VEGF agents thus can cause long-term, progressive atrophy of the neural retina, choroid, and RPE in patients with wet AMD.

An increase in the rate of progression of RPE atrophy has been reported in patients with wet AMD treated with anti-VEGF, which emphasizes the insufficient efficacy of this treatment in preventing the long-term visual consequences of AMD (Rosenfeld et al., 2011; Lois et al., 2013; Kumar et al., 2013; Grunwald et al., 2014; Young et al., 2014; Bhisitkul et al., 2015; Channa et al., 2015; Schütze et al., 2015).

Moreover, it has been shown that polymorphisms located in the FH gene lead to resistance in some patients treated with anti-VEGF antibody (Kloeckener-Gruissem et al., 2011; Brantley et al., 2007)

There is therefore a need to develop new therapies combining effectiveness and acceptability for the treatment of diseases involving neovascularization, particularly eye diseases. Surprisingly, for the first time the inventors have identified a novel function of FH. After extensive studies, they have demonstrated the ability of FH fragments to exert antiangiogenic activity. Such activity is particularly promising for the treatment of diseases involving neovascularization. Thus, in a first aspect, the invention relates to an FH fragment for use in the treatment and/or prophylaxis of a disease involving neovascularization, said FH fragment comprising the amino acid sequence represented in SEQ ID NO: 2.

The inventors have shown that FH fragments comprising SCR domain 7 at the very minimum, represented in SEQ ID NO: 2, inhibit neovascularization, in particular choroidal neovascularization. Also, factor H fragments appear to be a novel, effective, and relevant solution for treating diseases, particularly eye diseases, linked to excessive vascularization or choroidal neovascularization. The invention therefore provides a new therapeutic strategy which is effective in treating patients with eye diseases involving excessive vascularization or choroidal neovascularization.

“Factor H” or “FH” is understood to mean a glycoprotein involved in regulation of the alternative complement pathway. More specifically. FH plays a role in regulating the alternative complement pathway by interacting primarily with the C3b molecule. FH, encoded by the FH gene, is mainly synthesized in the liver but can also be locally expressed by different cell types including RPE cells, endothelial cells, and others. FH has repetitive sequences of approximately 60 amino acids called “SCR domains” (short consensus repeat). The region of FH involved in fluid phase regulation of complement alternative pathway activity is localized in domains SCR1-4. FH has binding sites for C3b (SCR1-4. SCR6-8. SCR12-14, and SCR19-20) and for glycosaminoglycans (GAGs) (SCR6-8 and SCR19-20). FH can thus interact with extracellular matrices. Domain SCR19-20 is also a binding site for C3b and specifically for C3d. FH also has binding sites for C-reactive protein or CRP (SCR6-8 and SCR 16-20), which is an acute-phase protein synthesized primarily in the liver and which plays an important role in inflammatory reactions and thus serves as a biomarker. Finally. FH has a binding site for Zinc (SCR6-8), important for the oligomerization of CFH. FH and Factor I (FI) regulate the alternative complement pathway by controlling the amplification loop of the alternative pathway. FH binds to C3b competitively with factor B (FB), which results in accelerated dissociation of the C3 (C3bBb) or C5 (C3bBbC3b) alternative convertase, which thus becomes inactive. In addition, the FH bound to C3b acts as co-factor for FI, enabling it to cleave C3b into C3bi, a molecule unable to bind to FB to form C3bBb. Deregulation or inactivation of the alternative pathway controlled by FH can result in uncontrolled activation of C3, resulting in assembly of the terminal components of the complement into membrane attack complex or MAC. This complex, which is composed of proteins of complement C5b9, remains bound and forms a transmembrane pore, a cytotoxic component of the complement system which causes lysis of target cells.

The prior art discloses that FH can be used to treat certain retinal degenerations due to its anti-inflammatory or antioxidant activity. Indeed, document WO2006/088950 discloses a polymorphism in the FH gene generating the Y402H variant that is associated with increased risk of developing AMD (Edwards et al., 2005; Hageman et al., 2005; Haines et al., 2005; Klein et al., 2005; Despriet et al., 2006). In addition, document WO2011/113641 describes the use of FH in the treatment and/or prophylaxis of several diseases, including eye diseases. This application describes an activity of FH which is based on its ability to bind to endogenous malondialdehyde, thus preventing the production of proinflammatory substances. Thus, the FI activity described therein is an anti-inflammatory activity: antiangiogenic activity of FI I is not described. In addition, the strategy proposed by document WO2011/113641 is for treating conditions which include an inflammatory component.

Document WO2013/140104 describes an antioxidant role for FH. In particular, it discloses that FH protects the RPE when exposed to oxidative stress, due to a protective effect on the tight junctions of the RPE cells against oxidative stress, specifically against destabilization and disruption of tight junctions of retinal cells exposed to oxidative stress. Again, the antiangiogenic activity of FI I is not described.

In the article by Kim et al. (2013), it is shown that human plasma FH has an antiangiogenic role in a model of laser-induced neovascularization in rats.

The present invention differs from these teachings by identifying the FH fragments for the first time, not the entire FH, having antiangiogenic activity.

The use of FH fragments, not the entire FH, has several advantages. The lower molecular weight of the fragments, in comparison to the entire protein, allows injecting a greater molar concentration of active molecules into the eye. The volumes of intraocular administration in AMD treatments are small (about 50 μl in humans). It is therefore advantageous to use smaller active substances, in order to increase their numbers when formulated in a low injection volume. In addition, the use of fragments limits the glycosylation sites, which allows limiting glycosylation variations during industrial production and thus ensuring structural uniformity when produced on a large scale. Furthermore, the inventors have show n that the FH fragments according to the invention not only decrease the neovascularization surface area (i.e. formation of new vessels) but also reinforce the membrane integrity of the new vessels, thereby reducing the release of liquid or blood.

The peptide sequence of native human FH is represented in SEQ ID NO: 1. This sequence is known to the skilled person and is available under accession number UniProtKB-P086031 in the UniProt database.

“FH fragment” is understood to mean a portion of the peptide sequence of Factor H. By definition, the term “fragment of factor H” excludes the factor H as a whole. Preferably, this FH fragment retains the same biological property of interest as human FH. In other words, it is a part of the peptide sequence of FH having the antiangiogenic property of FH. “Biological property of interest of the FH” is understood to mean the antiangiogenic property of said FH.

“FH fragment according to the invention” is understood to mean the FH fragments which at the very least comprise domain SCR 7 of human FH. The SCR7 sequence of human FH is represented in SEQ ID NO: 2. It is as follows:

CYFPYLENGYNQNYGRKFVQGKSIDVACHPGYALPKAQTTVTCMENGWSP
TPRC.

This sequence is encoded by the nucleotide sequence SEQ ID NO: 3.

In a preferred embodiment, the fragment of the invention preferably comprises domain SCR 7 fused to the N-terminal or C-terminal domains of FH.

In another preferred embodiment, said N-terminal or C-terminal domains of FH respectively consist of domains SCR 1-4 and SCR 19-20.

In another preferred embodiment, the fragment of the invention preferably comprises an amino acid sequence selected from among:
the sequence of domain SCR7 of human FH as represented in SEQ ID NO: 2 and encoded by nucleotide sequence SEQ ID NO: 3;
the sequence of domains SCR 1-7 of human FH as represented in SEQ ID NO: 4 and encoded by nucleotide sequence SEQ ID NO: 5:
the sequence of domains SCR 1-18 of human FH as represented in SEQ ID NO: 6 and encoded by nucleotide sequence SEQ ID NO: 7;
the sequence of domains SCR 7-20 of human FH as represented in SEQ ID NO: 8 and encoded by nucleotide sequence SEQ ID NO: 9:
the sequence of domains SCR 7-18 of human FH as represented in SEQ ID NO: 10 and encoded by nucleotide sequence SEQ ID NO: 11.
the sequence of domains SCR 6-8 of human FH as represented in SEQ ID NO: 12 and encoded by nucleotide sequence SEQ ID NO: 13.

“Polynucleotide of the invention” is understood to mean a nucleic acid sequence encoding a factor H fragment of the invention. Typically, the polynucleotide of the invention at the very least comprises a sequence encoding domain SCR7 of factor H as represented in SEQ ID NO: 3.

Preferably, the polynucleotide of the invention comprises a nucleic acid sequence selected from the group composed of SEQ ID NO: 5. SEQ ID NO: 7. SEQ ID NO: 9. SEQ ID NO: 1, and SEQ ID NO: 13.

For the first time, the inventors have identified a novel function of FH fragments as an antiangiogenic and the use of such an FH fragment for the treatment of eye diseases involving neovascularization. In the context of the present invention, the term “diseases involving neovascularization” includes all diseases characterized by abnormal angiogenesis activity. i.e. the formation of new blood vessels irrigating a tumor or any other tissue abnormalities, particularly ocular. In the invention, the terms neovascularization and angiogenesis are synonymous and refer to vascularization that is abnormal and/or excessive and/or likely to result in a pathological situation. Such neovascularization is found in several eye conditions, including wet AMD.

According to the invention, the term “treatment” or “treat” is understood to mean the act of removing, reducing, or inhibiting the progression of the disease to which the term is applied. The term “treatment” or “treat” is also understood to mean the act of removing, reducing, or inhibiting the progression of one or more symptoms of the condition or disease to which the term is applied.

According to the invention, the term “prophylaxis” or “prevention” is understood to mean preventing, delaying, or restricting the onset of a disease or the symptoms of a disease in a healthy subject or in a subject known to have a predisposition to said disease.

In a preferred embodiment, the disease involving neovascularization is an eye disease selected from among AMD, iris neovascularization, intraocular neovascularization, corneal neovascularization, retinal neovascularization, choroidal neovascularization, ischemic retinopathy, radiation retinopathy, neovascular glaucoma, diabetic retinopathy, retinopathy of prematurity, retinal vein occlusion, non-tumoral proliferative vascular diseases, and cancer.

In another preferred embodiment, the disease involving neovascularization is an eye disease selected from among AMD, iris neovascularization, intraocular neovascularization, corneal neovascularization, retinal neovascularization, choroidal neovascularization, and cancer.

In a preferred embodiment of the invention, said cancer is selected from the group composed of intraocular vascular and/or vascularized tumors, tumoral proliferative vascular diseases, vascular tumors of vascular or non-vascular origin such as choroidal hemangioma, retinal angiomatous proliferation, chorioretinal anastomosis, or polypoidal vasculopathy.

More preferably, the disease involving neovascularization is an eye disease selected from among choroidal neovascularization and AMD.

In a preferred embodiment of the invention, the disease involving neovascularization is neovascular AMD. This is a group of patients that are treatable by the antiangiogenic activity of the factor H fragments of the invention as disclosed by the inventors.

In a preferred embodiment of the invention, the disease involving neovascularization is choroidal neovascularization secondary to AMD or choroidal neovascularization secondary to pathological myopia, intraocular inflammation, or choroiditis.

“Age-related macular degeneration” or “AMD” is a disease which first affects the center of the retina, the macula, and thus causes a loss of clear vision. While peripheral vision is preserved, allowing those affected to orient themselves in general, AMD significantly affects the quality of life. It is the leading cause of irreversible loss of central vision in industrialized countries. Age is the primary risk factor for AMD. The RPE in the central portion of the retina is considered to be the first target of AMD. Damage or loss of function in the RPE leads to photoreceptor death. Various studies have shown that AMD is a complex disease associated with many factors, particularly environmental and genetic factors. There are two forms of AMD: dry form and wet form. The dry form, also called geographic atrophy or atrophic AMD, presents as a gradual disappearance of RPE cells and then of photoreceptors located in the macula. The wet form, also called neovascular or exudative, involves an angiogenesis process and results in proliferation of new abnormal blood vessels under the retina. These fragile vessels leak serum which is responsible for raising the retina, and/or blood resulting in the appearance of retinal hemorrhages.

“Choroidal neovascularization” corresponds to the appearance of choroidal neovessels. This corresponds to a proliferation of abnormal blood vessels under the retina, in the choroidal layer of the eyeball. The abnormal vessels can leak fluids and blood, causing edema and raising the retina. It most often involves:
metamorphopsia (abnormal vision characterized by deformation of images).
relative or absolute scotoma (i.e. a stationary gap in the visual field), and
predominantly near-field visual impairment.

Diagnosis is often confirmed by examinations consisting of fundus examination, fluorescein angiography, and optical coherence tomography. The choroidal neovascularization can be choroidal neovascularization secondary to AMD or choroidal neovascularization secondary to pathological myopia, to intraocular inflammation, or to infectious or non-infectious choroiditis.

It has been shown that MAC formation resulting from complement pathway activation is responsible for the development of laser-induced choroidal neovascularization in mice (Andreoli et al., 2009). MAC enables the release of angiogenic factors such as VEGF. PDGF, and FGF-2, resulting in amplification of the angiogenic process (Andreoli et al., 2009). MAC has also been shown to control the expression of VEGF in the CNV model in mice (Liu et al., 2011)

In a preferred embodiment, the invention relates to an amino acid sequence selected from the sequences represented in SEQ ID NO: 2. SEQ ID NO: 4. SEQ ID NO: 6. SEQ ID NO: 8. SEQ ID NO: 10, and SEQ ID NO: 12, for treating a condition among the following: AMD, preferably neovascular AMD, choroidal neovascularization, and cancer. All the technical characteristics defined above apply here.

The FH fragment according to the invention may be used alone, in combination, and/or in association with other active agents, such as other substances used in the treatment of eye diseases, in particular anti-VEGF agents, glucocorticoids, or biological agents such as anti-factor D. These various active agents may be used in combination therapy, and administered separately or in combination, spread out over time or concomitantly.

The FH fragment according to the invention may also be used for preparing a pharmaceutical composition for the treatment and/or prophylaxis of an eye disease involving neovascularization. Thus, the invention also relates to a pharmaceutical composition for use in the treatment and/or prophylaxis of an eye disease among the following: AMD, iris neovascularization, intraocular neovascularization, corneal neovascularization, retinal neovascularization, choroidal neovascularization, ischemic retinopathy, radiation retinopathy, neovascular glaucoma, diabetic retinopathy, retinopathy of prematurity, retinal vein occlusion, non-tumoral proliferative vascular disease, and cancer. All the characteristics mentioned above are applicable here.

The FH fragments according to the invention may be combined with any type of pharmaceutically acceptable carrier or excipient, and optionally with an extended-release matrix, such as biodegradable polymer, a biodegradable or non-biodegradable reservoir, particulate systems or intraocular or periocular implants for forming a pharmaceutical composition of the invention. The term “pharmaceutically acceptable” refers to molecular entities and compositions that do not cause an adverse, allergic, or otherwise unwanted reaction when administered to a mammal, particularly a human. A pharmaceutically acceptable carrier or excipient may be solid, semisolid, or liquid. The form of the pharmaceutical compositions, the route of administration, the dosage, and the dosage regimen naturally depend on the severity of the eye disease and its stage of development.

The pharmaceutical composition according to the invention may be in any form which can be administered to a patient, and in particular includes liquid or solid forms, typically eye drops, gel, or oral solution, but also suspensions, lyophilized powders, capsules, and tablets. Preferred are compositions formulated for local administration, such as eye drops and gels, or oral administration such as oral solutions, tablets, and ampoules. The pharmaceutical composition of the invention may also be in a form compatible with injection. i.e. local injection, administration through the mucosa, inhalation, oral administration, and more generally any formulation suitable for the intended purpose.

Preferably, the pharmaceutical composition according to the invention is in a form compatible for ocular, intraocular, intravitreal, sub-Tenon, subretinal, intra-orbital, subconjunctival, intravenous, intra-arterial, intramuscular, intraperitoneal, topical, subcutaneous, intranasal, oral, or parenteral administration.

The pharmaceutical composition according to the invention comprises from 10% to 90% by weight of FH fragment relative to the total weight of the composition. Preferably, the pharmaceutical composition of the invention contains from 20 to 60% of FH fragment relative to the total weight of the composition.

Among the modes of administration of a therapeutic protein to inside the eye, intravitreal injection is a simple way of releasing a protein into the posterior segment of the eye in order to minimize systemic exposure. However, repeated injection can cause complications such as hemorrhage, retinal detachment, or cataract.

To reduce the number of injections and to maintain the activity of the therapeutic protein at a constant level, other modes of administration have been developed. In particular, particulate or non-particulate polymers and particulate lipids (liposomes, micelles, nano-emulsions) allow gradual release of the encapsulated therapeutic protein, reducing toxicity and increasing its life. Finally, the pharmaceutical composition of the invention further comprises another active agent, for example such as other substances used in the treatment of eye diseases. Typically, the other active agent may be present in the pharmaceutical composition in an amount of at least 20% by weight relative to the total weight of said composition.

The factor H fragment according to the invention may be administered in the form of DNA or cDNA for local gene expression. Also, another object of the invention is a polynucleotide comprising the nucleic acid sequence represented in SEQ ID NO: 3, for use in the treatment and/or prophylaxis of a disease involving neovascularization.

In a preferred embodiment, said polynucleotide comprises a nucleic acid sequence selected from the group composed of the sequences represented in SEQ ID NO: 5. SEQ ID NO: 7. SEQ ID NO: 9. SEQ ID NO: 11, and SEQ ID NO: 13.

Another object of the invention is an expression vector comprising the polynucleotide encoding the factor H fragment of the invention, or an expression cassette comprising said polynucleotide for use in the treatment and/or prophylaxis of a disease involving neovascularization.

According to the invention, expression vectors suitable for use according to the invention may comprise at least one expression control element functionally linked to the nucleic acid sequence. The expression control elements are inserted into the vector and regulate expression of the nucleic acid sequence. Examples of expression control elements include lac systems, the phage lambda promoter, yeast promoters, and viral promoters. Other functional elements may be incorporated, such as a leader sequence, stop codons, polyadenylation signals, and sequences necessary for the transcription and subsequent translation of the nucleic acid sequence in the host system. It is understood by those skilled in the art that the correct combination of expression control elements depends on the chosen host system. It is also understood that the expression vector must contain the additional elements necessary for the transfer and subsequent replication of the expression vector containing the nucleic acid sequence in the host system. Such vectors are easily constructed using conventional or commercially available methods.

Expression of the polynucleotide of the invention may for example be carried out locally by any method for transferring the vectored polynucleotide by viral or nonviral methods in any cell of the eye. The invention therefore relates to a composition comprising a polynucleotide according to the invention for use as a gene therapy drug. Means by which the vector carrying the polynucleotide can be introduced into cells include microinjection, electroporation, transduction or transfection using DEAE-dextran, lipofection, calcium phosphate, or other procedures known to the skilled person.

In a preferred embodiment, eukaryotic expression vectors that function in eukaryotic cells are used. Examples of such vectors include viral vectors such as retrovirus, adenovirus, herpes virus, vaccinia virus, smallpox virus, poliovirus, lentivirus, bacterial expression vectors, or plasmids such as pcDNA5.

Viral vectors offer the advantage of good transfection efficiency but do not control the amount of proteins secreted. The encapsulated cell technology was developed in order to administer a therapeutic protein in a controlled, sustained, and extended manner in the posterior segment of the eye. Also, electroporation techniques for specific transfection of the ocular ciliary muscle make it possible to use the ciliary muscle as a reservoir of plasmids encoding therapeutic proteins and thus to express and secrete the fragment of the invention intraocularly and over an extended period.

In another aspect, the invention relates to a therapeutic method for treating a disease involving neovascularization, said method comprising administration of a factor H fragment comprising the amino acid sequence represented in SEQ ID NO: 2. All the technical characteristics mentioned above are applicable here.

In another aspect, the invention relates to the use of a factor H fragment comprising the amino acid sequence represented in SEQ ID NO: 2, to obtain a drug for treating a disease involving neovascularization. All the technical characteristics mentioned above are applicable here. The following examples are illustrative only, and do not limit the scope of the invention.

### EXAMPLE

Here the inventors demonstrated the therapeutic potential of FH fragments in relevant models of diseases involving neovascularization. To do so, they produced several FH fragments in cultured cells. They then purified these fragments. Quantitation of the biological activity of the fragments was tested in vitro concerning the ability of FH fragments: (1) to inhibit the lysis of sheep erythrocytes induced by the alternative complement pathway (activity related to the C5b9 complex). (2) to accelerate the dissociation of the C3 convertase complex previously formed in 96-well ELISA plates (anti-C3 convertase activity). (3) to inactivate, in vitro, the molecule of the C3b complement by acting as a cofactor for factor I (FI). Finally, the inventors measured the inhibition of angiogenesis by the fragments produced in the laser-induced CNV model in rats. This study, unexpectedly and for the first time, revealed an antiangiogenic role of FH fragments.

### I. Preparation of Factor H Fragments

The inventors have developed FH fragments comprising various domains.
the sequence of domains SCR 1 to 7 of human FH as represented in SEQ ID NO: 4, and encoded by nucleotide sequence SEQ ID NO: 5:
the sequence of domains SCR 1 to 18 of human FH as represented in SEQ ID NO: 6, and encoded by nucleotide sequence SEQ ID NO: 7;
the sequence of domains SCR 7 to 20 of human FH as represented in SEQ ID NO: 8, and encoded by nucleotide sequence SEQ ID NO: 9:
the sequence of domains SCR 7 to 18 of human FH as represented in SEQ ID NO: 10 and encoded by nucleotide sequence SEQ ID NO: 11.
the sequence of domains SCR 6 to 8 of human FH as represented in SEQ ID NO: 12 and encoded b) nucleotide sequence SEQ ID NO: 13.
the sequence of domains SCR 1 to 4 of human FH as represented in SEQ ID NO: 14 and encoded by nucleotide sequence SEQ ID NO: 15;
the sequence of domains SCR 1 to 6 of human FH as represented in SEQ ID NO: 16 and encoded by nucleotide sequence SEQ ID NO: 17:
the sequence of domains SCR 8 to 20 of human FH as represented in SEQ ID NO: 18 and encoded by nucleotide sequence SEQ ID NO: 19;

The name, nature and sequence of these fragments are summarized in Table 1 below:

| | SCR of the FH | | |
|---|---|---|---|
| Name of | contained | Amino acid | |
| fragment | in the fragment | sequence | Comments |
| “1NT7” | SCR 1 to 7 | SEQ ID NO: 4 | Fragment of the |
| | | | invention |
| “1NT18” | SCR 1 to 18, | SEQ ID NO: 6 | Fragment of the |
| | | | invention |
| “7CT20” | SCR 7 to 20 | SEQ ID NO: 8 | Fragment of the |
| | | | invention |
| “7CT18” | SCR 7 to 18 | SEQ ID NO: 10 | Fragment of the |
| | | | invention |
| “SCR6-8” | SCR 6 to 8 | SEQ ID NO: 12 | Fragment of the |
| | | | invention |
| “SCR1-4” | SCR 1 to 4 | SEQ ID NO: 14 | Comparative fragment |
| “SCR1-6” | SCR 1 to 6 | SEQ ID NO: 16 | Comparative fragment |
| “8CT20 | SCR 8 to 20 | SEQ ID NO: 18 | Comparative fragment |

### II. Production and Purification of Factor H Fragments

The nucleic acid sequences of the FH fragments were cloned in a plasmid vector enabling expression of these molecules in eukaryotic cells. Secretion of these molecules in the cell culture supernatant was facilitated by using a suitably chosen signal peptide (MB7) in the N-terminal position. The fragments were produced by transient transfection into the HEK 293F human cell line. After 7 days of production in batch mode, the supernatant was harvested, clarified, concentrated, and sterile filtered (0.22 μm).

Purification of the FH fragments was carried out by affinity chromatography on a Ni-NTA column or cobalt column via the hexahistidine tag added at the C-terminus of the FH fragments. The FH fragment INT18 not containing a tag at the C-terminus was purified by one or two steps of ion exchange chromatography (SP-Sepharose and Q-Sepharose). The eluted FH fragments were dialyzed against PBS and concentrated if the final concentration determined by absorbance at 280 nm was less than 30 μg/ml.

### III. In Vitro Characterization of Functional Activity of FH Fragments

### Material and Methods

The anti-C3 convertase activity, expressed as EC50 values, determines the concentration of F I or FH fragments required to dissociate 50% of the C3 convertase (C3bBb) preformed (IC50) after 32 min incubation at 34° C. In a first step, the generation of C3 convertase is therefore carried out in the wells of a microtiter plate as follows: 100 μl of a solution of C3b (2.5 μg/ml) are deposited per well and incubated overnight at 4° C.; after saturation of unoccupied binding sites, the step of generating C3 convertase is accomplished by adding 4 μg/ml factor B (FB), 0.3 μg/ml factor D (FD), and 1.5 mM NiCl2, and incubating the mixture at 34° C. for 120 minutes. In a second step, the C3 convertase formed is dissociated by addition of whole FH or FH fragments at different concentrations for 32 to 34 min at 34° C. After washing, the amount of C3 convertase complex still present is determined b) detecting human FB using a goat anti-FB antibody and a goat anti-Ig antibody (H+L) that is peroxidase-coupled.

The FH, which is in fluid phase, acts as a cofactor of FI which cleaves C3b. FI cuts the 110 kDa α′-chain of the C3b, releasing a 4d kDa α′ fragment and thus produces C3bi. The FH or its fragments are incubated at different amounts with factor 1 and C3b purified (0.14 μg and 10 μg, respectively) at 37° C. for 30 minutes. The reaction is quenched by adding a denaturation/reduction buffer containing SDS and incubating for 3 minutes at 95° C. Control reaction mixtures containing either C3b alone or the C3b and FI mixture are also prepared and incubated under the same conditions. The samples are then loaded onto 10% polyacrylamide gel in the presence of SDS and subjected to electrophoresis (SDS-PAGE). After staining the gels with Coomassie blue, colored bands corresponding to the products from cleaving the α′ chain are scanned, quantified by densitometric analysis, and normalized to the values obtained for the uncleaved α′ chain.

Sheep red blood cells having a surface rich in sialic acid bind FH by its C-terminal portion, preventing the formation of C3 convertase and therefore cell lysis. A suspension of sheep red blood cells (1×10⁸ SRBC/ml) is taken up in a reaction buffer containing 10 mM EGTA and 7 mM MgCl2 and exposed to a mixture of normal pooled plasma and Fit-depleted plasma to allow activation of the alternative complement pathway. Increasing concentrations of FH or FH fragments are added before inducing activation of the alternative complement pathway in order to evaluate SRBC lysis inhibition by these molecules. The mixture is incubated at 37° C. in a water bath for 30 to 35 minutes, then the reaction is quenched b) adding 10 mM Hepes buffer containing 2 mM EDTA.

After centrifugation for 5 minutes at 1730 g, 200 μl supernatants are collected in order to measure the optical density at 414 nm. Measurement of antihemolytic activity is determined as a % of the control, its value corresponding to the molar concentration of plasma FH required to obtain complete inhibition of lysis (100%).

### Results

TABLE 2
Biological activities of FH fragments

| | Molecular | Anti-C3 | | Antihemolytic |
|---|---|---|---|---|
| FH | weight | convertase | F1 cofactor | activity |
| fragment | (Da) | (EC₅₀ μg/ml) | (%) | (%) |
| “1NT7” | 49 927 | 0.038-0.078 | 100% | 93% |
| “1NT18” | 122 335 | 0.129-0.1566 | 100% | 0% |
| “7CT20” | 96 281 | ∞ | 0% | |
| “7CT18” | 96 281 | ∞ | 0% | 16% |
| “SCR6-8” | 21 832 | ∞ | 0% | 8.5% |
| “1NT4” | 29 197 | 1.88-4.37 | 100% | 100% |
| “1NT6” | 43267 | 0.093 | 100% | 0% |
| “8CT20” | 89 596 | ∞ | 0% | |

The results presented in the above table show that:
only fragments containing SCR 1 to 4 have anti-C3 convertase activity. In addition, the activity of the minimal fragment “INT4” is significantly lower than that of the fragments containing the subsequent SCRs (SCR 5-18).
similarly, only the fragments containing SCR 1 to 4 have FI cofactor activity.
the antihemolytic activity is carried by fragments composed of SCR 1 to 4 for inhibiting C3 convertase activity and SCR 19-20 for binding to sialic acid residues present on the surface of the sheep red blood cells.

### IV. Analysis of Angioenesis Inhibition

The inventors evaluated the antiangiogenic activity induced by each of the FH fragments described in section I. To do this, they used an in vivo model of choroidal neovascularization (CNV) in Long-Evans rats (Janvier Labs. Le Genest-Saint-Isle. France).

### Materials and Methods

### 1. Quantitative Assay for Determining Choroidal Neovascularization.

The rats were anesthetized (using a mixture of ketamine/xylazine at a ratio of 100 μl/100 mg), and the eyes were dilated with Mydriaticum and anesthetized with tetracaine. Six Argon laser impacts on two papillae of the optic nerve were carried out at regular intervals. Next, the cornea of the rats was protected by application of “Goniosol” gel. Then 3 μl of saline alone or a dilute FH solution in saline were injected into the vitreous body of the eyes of each rat. Two injection times were tested: 1) preventative on the same day as the laser, and 2) corrective, four days post-laser. After sacrifice on days 7 or 14 post-laser, the eyes of the rats were enucleated and fixed in paraformaldehyde solution. Only the posterior part of the eye (RPE/choroid/sclera) was collected and then radially sliced. After blocking the nonspecific sites with a goat serum solution, the RPE/choroid/sclera complexes were labeled with a marker of endothelial cells diluted in saline solution. The RPE/choroid/sclera complexes were then mounted between slide and cover slip using a mounting medium. The markers were observed using a Zeiss confocal microscope and images of the set of laser impacts, called stacks, were collected. These images were then analyzed using “ImageJ” software, quantifying the labeling surface for each impact and for each rat eye. For each experiment, at least 3 groups of 4 rats were tested: I) uninjected rats (control group), 2) rats injected with saline solution (injection test group), and 3) rats injected with FH solution.

### Results

The inventors measured the inhibition of choroidal neovascularization provided by the different FH fragments produced.

These results are summarized in Table 3 below:

| | SCR of the FH | | Inhibition of | |
|---|---|---|---|---|
| Name of | contained in the | Amino acid | choroidal | Inhibition of |
| fragment | fragment | sequence | neovascularization | MAC formation |
| Whole FH | SCR 1 to 20 | SEQ ID NO: 1 | 76% | 85% |
| “1NT7” | SCR 1 to 7 | SEQ ID NO: 4 | 73% | 69% |
| “1NT18” | SCR 1 to 18, | SEQ ID NO: 6 | 67% | 59% |
| “7CT20” | SCR 7 to 20 | SEQ ID NO: 8 | 80% | 63% |
| “7CT18” | SCR 7 to 18 | SEQ ID NO: 10 | 56% | 88% |
| “SCR6-8” | SCR 6 to 8 | SEQ ID NO: 12 | 0% | 85% |
| “SCR1-4” | SCR 1 to 4 | SEQ ID NO: 14 | 0% | 15% |
| “SCR1-6” | SCR 1 to 6 | SEQ ID NO: 16 | 47% | 40% |
| “8CT20 | SCR 8 to 20 | SEQ ID NO: 18 | 0% | 0% |

These results clearly show that the fragments of the invention, meaning the fragments comprising domain SCR7 of FH, provide maximum inhibition of choroidal neovascularization.

Fragments providing inhibition of choroidal neovascularization also act to inhibit MAC formation.

To the inventors' knowledge, these are the first tests demonstrating an antiangiogenic role of FH fragments comprising domain SCR7.

These fragments appear particularly relevant to the development of treatment strategies for diseases involving neovascularization.

### V—Alteration of CNV Gene Signature by FH Fragments

To assess the activity of FH and its fragments on the CNV gene signature, quantitative analysis of the expression of genes encoding the molecules for angiogenesis in the retina/pigment epithelium cells/choroid complex was performed using the qRT-PCR method.

### Results

The results show that the rFHINT7 and rFH7CT20 fragments have the same inhibitory activity on the transcription of angiogenesis genes, namely VEGFA. VEGFR1, and VEGFR2, as the entire rFH. In addition, the entire FH and the two fragments induce increased expression of the gene encoding PEDF, an angiogenesis inhibitory factor.

1. A method of treatment and/or prophylaxis of a disease involving neovascularization in a subject in need thereof comprising the step of administering to the subject a Factor H fragment comprising the amino acid sequence represented in SEQ ID NO: 2, fused to N-terminal and/or C-terminal domains of Factor H.

2. The method according to claim 1, wherein the Factor H fragment comprising the amino acid sequence represented in SEQ ID NO: 2 is fused to domains SCR 1-4 of human factor H and/or domains SCR 19-20 of factor H.

3. The method according to claim 1, wherein the Factor H fragment comprising the amino acid sequence represented in SEQ ID NO: 2 is selected from the group consisting of SEQ ID NO: 4, SEQ ID NO: 6, SEQ ID NO: 8, and SEQ ID NO: 10.

4. The method according to claim 1, wherein the disease involving neovascularization is selected from the group consisting of AMD, iris neovascularization, intraocular neovascularization, corneal neovascularization, retinal neovascularization, choroidal neovascularization, ischemic retinopathy, radiation retinopathy, neovascular glaucoma, diabetic retinopathy, retinopathy of prematurity, retinal vein occlusion, and non-tumoral proliferative vascular diseases and cancer.

5. The method according to claim 4, wherein the cancer is selected from the group consisting of intraocular vascular and/or vascularized tumors, tumoral proliferative vascular diseases, vascular tumors of vascular or non-vascular origin such as choroidal hemangioma, angiomatous intraretinal proliferation, chorioretinal anastomosis, and polypoidal vasculopathy.

6. The method according to claim 1, wherein the disease involving neovascularization is an eye disease selected from the group consisting of:
neovascular AMD,
choroidal neovascularization, and
ocular tumors of vascular or non-vascular origin.

7. The method according to claim 2, wherein the disease involving neovascularization is choroidal neovascularization secondary to AMD or choroidal neovascularization secondary to pathological myopia, intraocular inflammation, or infectious or non-infectious choroiditis.

8. A method of treatment and/or prophylaxis of an eye disease selected from the group consisting of AMD, iris neovascularization, intraocular neovascularization, corneal neovascularization, retinal neovascularization, choroidal neovascularization, ischemic retinopathy, radiation retinopathy, neovascular glaucoma, diabetic retinopathy, retinopathy of prematurity, retinal vein occlusion, non-tumoral proliferative vascular disease, and cancer in a subject in need thereof comprising the step of administering to the subject a pharmaceutical composition comprising a factor H fragment comprising the amino acid sequence represented in SEQ ID NO: 2 fused to N-terminal and/or C-terminal domains of factor H.

9. Method according to claim 8, wherein said cancer is selected from the group consisting of intraocular vascular and/or vascularized tumors, tumoral proliferative vascular diseases, vascular tumors of vascular or non-vascular origin such as choroidal hemangioma, retinal angiomatous proliferation, chorioretinal anastomosis, and polypoidal vasculopathy.

10. Method of treatment and/or prophylaxis of a disease involving neovascularization in a subject in need thereof comprising the step of administering to the subject a polynucleotide comprising the nucleic acid sequence represented in SEQ ID NO: 3.

11. The method according to claim 10, wherein said polynucleotide comprises a nucleic acid sequence selected from the group consisting of the sequences represented in SEQ ID NO: 5, SEQ ID NO: 7, SEQ ID NO: 9, and SEQ ID NO: 11.

12. Method of treatment and/or prophylaxis of a disease involving neovascularization in a subject in need thereof comprising the step of administering to the subject an expression vector comprising the the nucleic acid sequence represented in SEQ ID NO: 3.

13. (canceled)
